(19) United States
(12) Patent Application Publication
(10) Pub. No.: US 2025/0329486 A1
(43) Pub. Date: Oct. 23, 2025

(54) Vector Potential Coil Device, Vector Potential Generation Device, And Treatment Device

(71) Applicants: SUMIDA CORPORATION, Tokyo (JP); NATIONAL UNIVERSITY CORPORATION, IWATE UNIVERSITY, Iwate (JP)

(72) Inventors: Masaki SAITO, Natori City (JP); Masahiro DAIBO, Morioka-shi (JP)

(21) Appl. No.: 19/258,792

(22) Filed: Jul. 2, 2025

Related U.S. Application Data
(63) Continuation of application No. PCT/JP2023/030578, filed on Aug. 24, 2023.

(30) Foreign Application Priority Data
Jan. 12, 2023 (JP) 2023-003289

(51) Int. Cl. H01F 7/20; A61N 2/00; A61N 2/02; H01F 27/28

(52) U.S. Cl. CPC H01F 7/20; A61N 2/006; A61N 2/02; H01F 27/28

(57) ABSTRACT

A vector potential coil device includes a layered conductor member in a spiral roll shape, a first end surface part on an inner circumferential side of a roll of the layered conductor member, and a second end surface part on an outer circumferential side of a roll of the layered conductor member. A power supply device conducts a current to the layered conductor member via the first end surface part and second end surface part to generate a vector potential in the layered conductor member.

### CROSS REFERENCE TO RELATED APPLICATIONS

This application is a continuation of PCT Application No. PCT/JP2023/030578, filed on Aug. 24, 2023, which claims priority to Japanese Patent Application No. 2023-003289, filed on Jan. 12, 2023. The contents of both of the above applications are expressly incorporated herein by reference in their entirety.

### BACKGROUND

### Technical Field

The present invention relates to a vector potential coil device, a vector potential generation device, and a treatment device.

### Background Art

One vector potential generation device generates a vector potential by using a solenoid coil that extends along a coil axis that circulates around in a helical shape. For instance, refer to International Patent Publication Number WO2015/099147.

In the above-mentioned vector potential generation device, the solenoid coil that is wound with a small coil diameter extends along the coil axis that circulates around in the helical shape. Therefore, a conducting wire, which is configured with the solenoid coil, is long, and at the same time, the conducting wire itself is thin. As a result, the resistance and inductance of the solenoid coil are increased. As mentioned above, when the resistance and inductance become large, it becomes difficult to conduct a large alternating current through the solenoid coil, and ultimately, it becomes difficult to generate a strong vector potential.

### SUMMARY

The present invention has been made in view of the above and has an object that is to obtain a vector potential coil device capable of conducting a large current, a vector potential generation device that generates a vector potential by using such the vector potential coil device, and a treatment device that has such the vector potential generation device.

A vector potential coil device according to the present invention has a layered conductor member being formed in a spiral roll shape, a first end surface part on an inner circumference side of the roll of the layered conductor member, and a second end surface part on an outer circumference side of the roll of the layered conductor member. Further, the layered conductor member generates a vector potential by a current being conducted through the first and second end surface parts.

A vector potential generation device according to the present invention has the vector potential coil device that is mentioned above and a power supply device that conducts a current through the vector potential coil device.

A treatment device according to the present invention has the vector potential generation device that is mentioned above and a controller that controls the power supply device of the vector potential generation device. In addition, the vector potential coil device applies a vector potential to a living body.

### Effects of the Invention

According to the present invention, it is possible to obtain a vector potential coil device capable of conducting a large current, a vector potential generation device that generates a vector potential by using such the vector potential coil device, and a treatment device that has such the vector potential generation device.

### BRIEF DESCRIPTION OF THE DRAWINGS

FIG. 1 is a perspective view that shows a configuration of a vector potential generation device according to a first embodiment of the present invention.

FIG. 2 is a side view that shows a vector potential coil device 1 shown in FIG. 1.

FIG. 3 is a development view of a layered conductor member 11 shown in FIG. 1.

FIG. 4 is a development view that shows the layered conductor member 11 of the vector potential coil device 1 according to a second embodiment.

FIG. 5 is a diagram that shows an equivalent circuit of the vector potential coil device 1 shown in FIG. 4.

FIG. 6 is a perspective view that shows a configuration of a vector potential generation device according to a third embodiment of the present invention.

FIG. 7 is a diagram that shows an arrangement example of the vector potential generation device shown in FIG. 6.

### DETAILED DESCRIPTION

Embodiments of the present invention will be explained below with reference to the drawings.

### First Embodiment

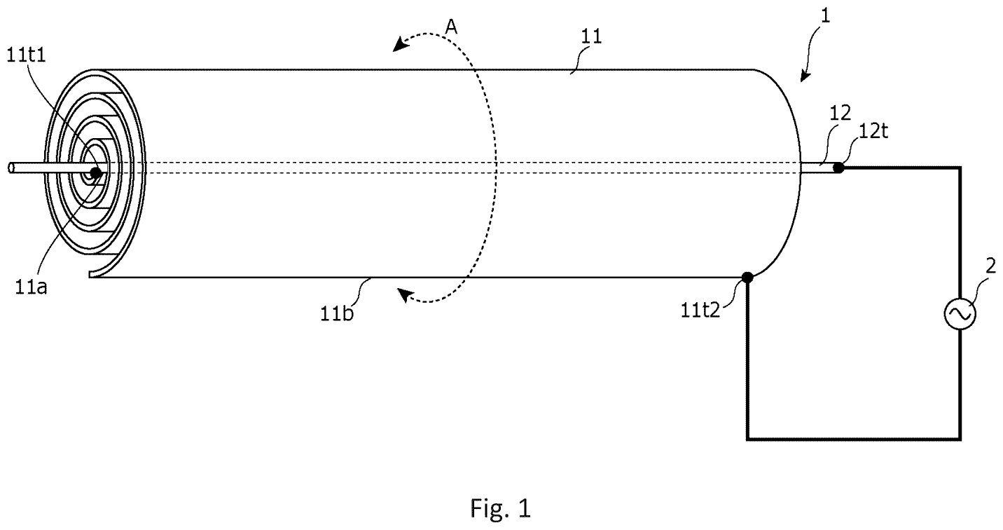

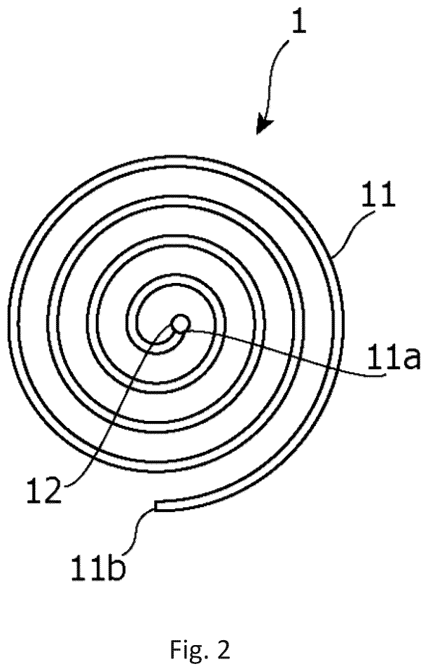

FIG. 1 is a perspective view that shows a configuration of a vector potential generation device according to a first embodiment of the present invention. FIG. 2 is a side view that shows a vector potential coil device 1 shown in FIG. 1. The vector potential generation device shown in FIG. 1 has the vector potential coil device 1 and a power supply device 2.

As shown in FIGS. 1 and 2, the vector potential coil device 1 has a layered conductor member 11 being formed in a spiral roll shape. The layered conductor member 11 has a first end surface part 11a that is an end surface on an inner circumference side (inner peripheral side) of the roll, and a second end surface part 11b that is an end surface on an outer circumference side (outer peripheral side) of the roll. Here, the layered conductor is a non-magnetic member having excellent member 11 electrical conductivity (for instance, a copper member/material).

In the first embodiment, the layered conductor member 11 is formed in the spiral roll shape centered on a linear central axis.

In addition, the power supply device 2 generates a voltage of a specific waveform and conducts a current of a specific waveform varying in time (with time variation) (for instance, an alternating current such as a sine wave, a triangular wave, or a rectangular wave, a pulse current, or a current combining these currents) to the layered conductor member 11 via the first end surface part 11a and the second end surface part 11b. For instance, the power supply device 2 is a high frequency power source of about 1 kHz to 1 GHz. Further, for instance, the power supply device 2 may be configured to continuously conduct an AC current (alternating current) of a predetermined frequency through the layered conductor member 11, or may be configured to conduct the AC current of a predetermined frequency intermittently at a predetermined time interval or intermittently in a predetermined time series pattern through the layered conductor member 11.

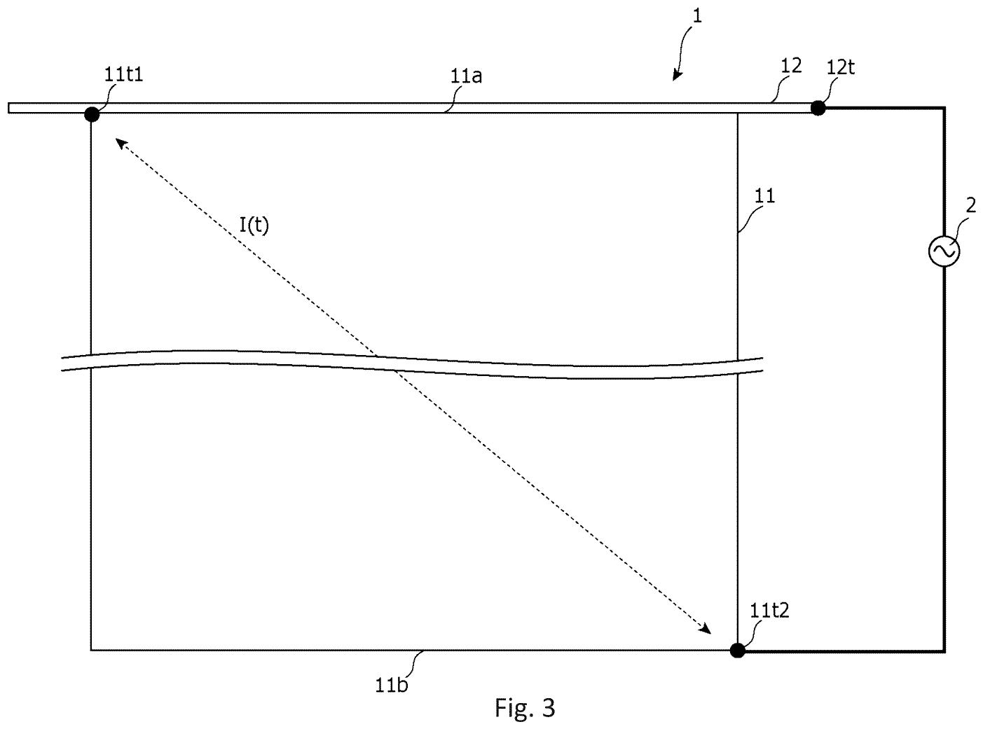

FIG. 3 is a development view of the layered conductor member 11 shown in FIG. 1. As shown in FIG. 3, in the first embodiment, the layered conductor member 11 is a substantially rectangular plate member.

Furthermore, as shown in FIGS. 1 to 3, the vector potential coil device 1 further has a core conductor member 12 that is connected (electrically and mechanically) to the first end surface part 11a along the first end surface part 11a. The core conductor member 12 is a rod-shaped member and has, for instance, a diameter larger than a thickness of the layered conductor member 11. In addition, the power supply device 2 is electrically connected to the core conductor member 12 and conducts the above-mentioned current I(t) of the specific waveform varying in time through the layered conductor member 11 via the core conductor member 12, the first end surface part 11a, and the second end surface part 11b.

Specifically, here, as shown in FIG. 3, one end of output terminals of the power supply device 2 is electrically connected to one end 12t of the core conductor member 12. Further, an electrical connection point between the core conductor member 12 and the layered conductor member 11 is provided at an end 11t1, of both ends of the first end surface part 11a, on the other end side of the core conductor member 12. In addition, the other end of the output terminals of the power supply device 2 is electrically connected to an end 11t2 of the second end surface part 11b of the layered conductor member 11 that is diagonal to the end 11t1. Note that the core conductor member 12 and the layered conductor member 11 are not electrically connected to each other at any portion other than the end 11t1. As a result, the current I(t) is conducted through the layered conductor member 11 between the end 11t1 and the end 11t2.

The above-mentioned current I(t) flows in a spiral pattern around the central axis (of the spiral roll shape). Therefore, when the current I(t) is an AC current, an alternating magnetic field is generated in a direction of the central axis, and an alternating vector potential A circulating with the central axis as the center is generated.

In the first embodiment, the core conductor member 12 is formed with a soft magnetic material such as permalloy and has soft magnetism (a soft magnetic property). As a result, the above-mentioned AC magnetic field (alternating magnetic field) is enhanced, and ultimately, the vector potential A is also enhanced.

Next, an operation of the above-mentioned vector potential generation device will be explained.

The power supply device 2 applies an AC voltage of a predetermined frequency to the end 12t of the core conductor member 12 and the second end surface part 11b of the layered conductor member 11 so as to conduct the current I(t) through the layered conductor member 11 via the core conductor member 12, the first end surface part 11a (the end 11t1), and the second end surface part 11b (the end 11t2).

In the layered conductor member 11, the current I(t) flows in the spiral pattern between the first end surface part 11a and the second end surface part 11b. As a result, when the current I(t) is an alternating current (AC) current, an alternating current (AC) magnetic field is generated along the central axis (the core conductor member 12) (of the spiral roll shape), and at the same time, an alternating current (AC) vector potential A (alternating vector potential A) circulating around the central axis is generated.

In this case, the greater an axial length of the layered conductor member 11 is, the greater a width in a direction substantially perpendicular to the conducting direction of the current I(t) becomes, and the smaller a resistance value between the first end surface part 11a and the second end surface part 11b becomes. Therefore, a large current can be conducted through the layered conductor member 11, and ultimately, a large vector potential A can be generated.

As mentioned above, according to the first embodiment, the vector potential coil device 1 has the layered conductor member 11 being formed in the spiral roll shape, the first end surface part 11a on the inner circumference side of the roll of the layered conductor member 11, and the second end surface part 11b on the outer circumference side of the roll of the layered conductor member 11. The power supply device 2 conducts the current I(t) through the layered conductor member 11 via the first end surface part 11a and the second end surface part 11b, thereby generating the vector potential in the layered conductor member 11.

As a result, a large current can be conducted through the vector potential coil device 1 and can obtain a high-output vector potential generation device. Therefore, for instance, it is expected that an application of a strong vector potential to a head can be used to treat brain diseases, such as epilepsy.

Further, by providing the core conductor member 12 having soft magnetism as mentioned above, a stray capacitance becomes relatively small. As a result, the characteristics of the vector potential coil device 1 at a high frequency region become excellent.

Furthermore, since the layered conductor member 11 can be formed, for instance, by winding a plate-shaped metal member into a spiral roll shape, the vector potential coil device 1 can be manufactured relatively easily.

### Second Embodiment

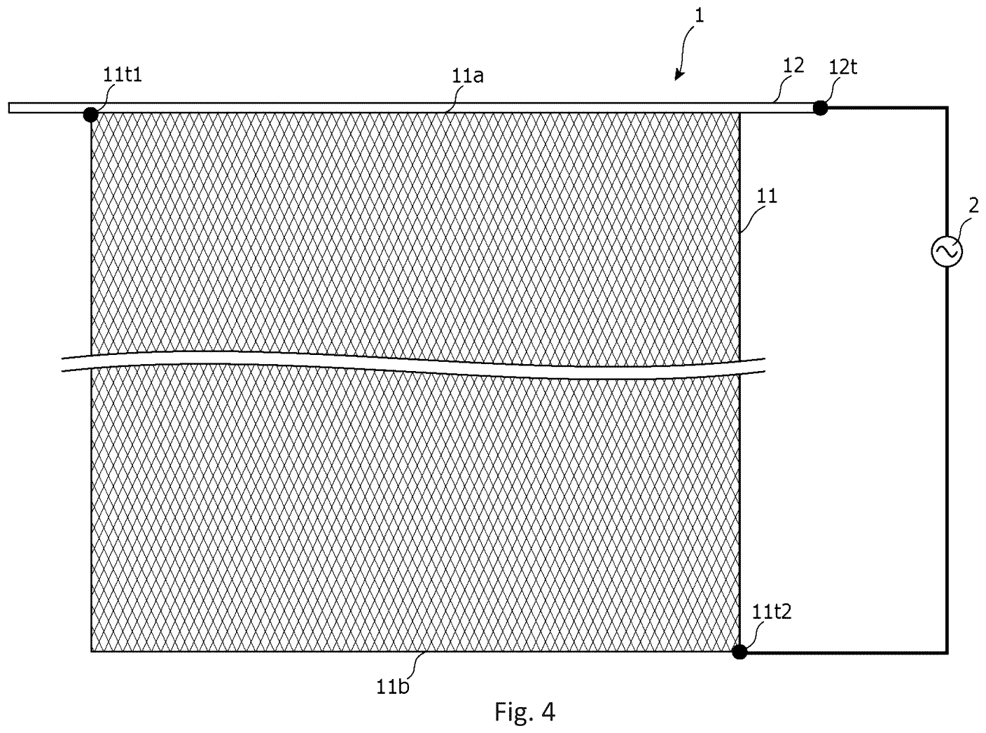

FIG. 4 is a development view that shows a layered conductor member 11 of a vector potential coil device 1 according to a second embodiment.

In the second embodiment, as shown in, for instance, FIG. 4, the layered conductor member 11 is a mesh member (a mesh-like member, a netlike member, a vascular member, a network member, or a reticulated member). For instance, the layered conductor member 11 is made by connecting a plurality of copper wires to one another in a net-like shape. Note that the net-like shape (mesh shape) of the layered conductor member 11 is not limited to the configuration shown in FIG. 4. In addition, the mesh member used for the layered conductor member 11 may be a plate member having a plurality of holes such as a punched metal (perforated metal).

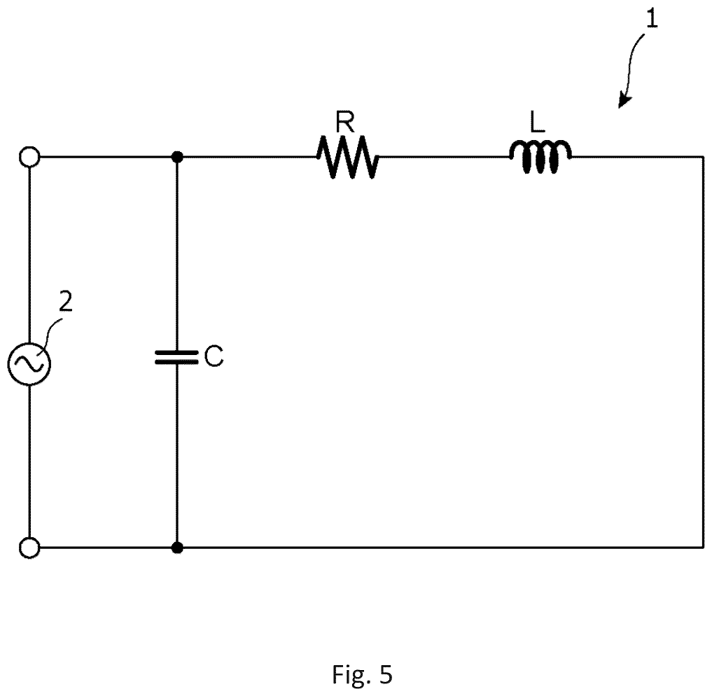

FIG. 5 is a diagram that shows an equivalent circuit of the vector potential coil device 1 shown in FIG. 4. As shown in FIG. 5, the vector potential coil device 1, when viewed from the power supply device 2, can be considered as a circuit of a resistance R, an inductance L, and a stray capacitance C. The resistance R is the resistance between the first end surface part 11a and the second end surface part 11b. The inductance L is the inductance between the first end surface part 11a and the second end surface part 11b.

Here, as compared with a vector potential coil device in which a solenoid coil being wound with a small coil diameter extends along a coil axis that circulates around in a spiral shape, in the vector potential coil device 1 in the second embodiment, the axial length of the layered conductor member 11 is great, the width in the direction substantially perpendicular to the conducting direction of the current is great, and the number of rolls is relatively small. As a result, the resistance R and the inductance L are small. Further, since the layered conductor member 11 is the mesh member, the opposing area between the layers of the layered conductor member 11 is small. Therefore, the stray capacitance C is also small. As a result, a large current can be conducted.

Furthermore, in the vector potential coil device 1 in the second embodiment, since the layered conductor member 11 is the mesh member, a conductor surface area is large, and an influence of a skin effect is small even with the high frequency AC current. Further, since the layered conductor member 11 has excellent heat dissipation characteristics, it is possible to conduct a large current.

Note that the other configurations and operations of the vector potential generation device according to the second embodiment are the same as those explained in the first embodiment. Therefore, the explanations of them will be omitted.

### Third Embodiment

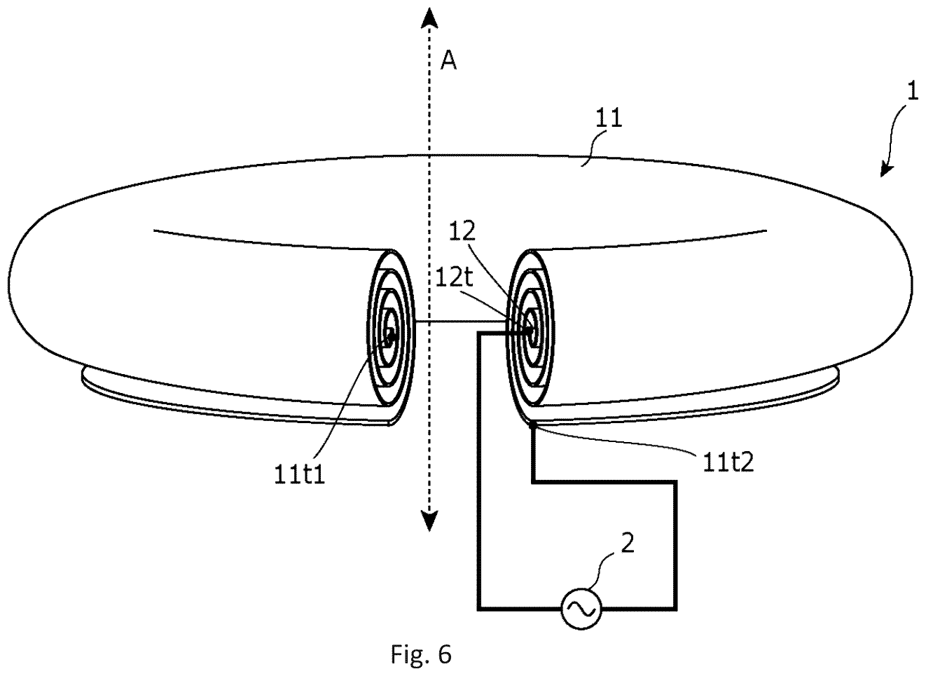

FIG. 6 is a perspective view that shows a configuration of a vector potential generation device according to a third embodiment of the present invention.

In the third embodiment, as shown in, for instance, FIG. 6, a layered conductor member 11 is formed in a spiral roll shape centered on a curved central axis.

Here, as shown in, for instance, FIG. 6, the layered conductor member 11 is formed in the spiral roll shape centered on a ring-shaped (circular-shaped) central axis. The outer shape of the layered conductor member 11 is formed in a substantially torus shape. Further, a vector potential A is generated so as to interlink with (or cross) the substantially torus shape.

In addition, in FIG. 6, the outer shape of the layered conductor member 11 is formed in the substantially torus shape. However, the outer shape of the layered conductor member 11 may also be formed in a spiral roll shape centered on an arc-shaped central axis in which, for example, the half of the circumference of the substantially torus outer shape is cut along the circumferential direction.

Further, the layered conductor member 11 in the third embodiment may be the plate member (without holes) as the first embodiment, or may also be the mesh member as the second embodiment.

Note that the other configurations and operations of the vector potential generation device according to the third embodiment are the same as those explained in the first embodiment or the second embodiment. Therefore, the explanations of them will be omitted.

### Fourth Embodiment

A treatment device according to a fourth embodiment of the present invention has a vector potential generation device according to any of the first to third embodiments mentioned above, and applies a vector potential generated by these vector potential generation devices to a specific part of a living body (for instance, a human body or an animal).

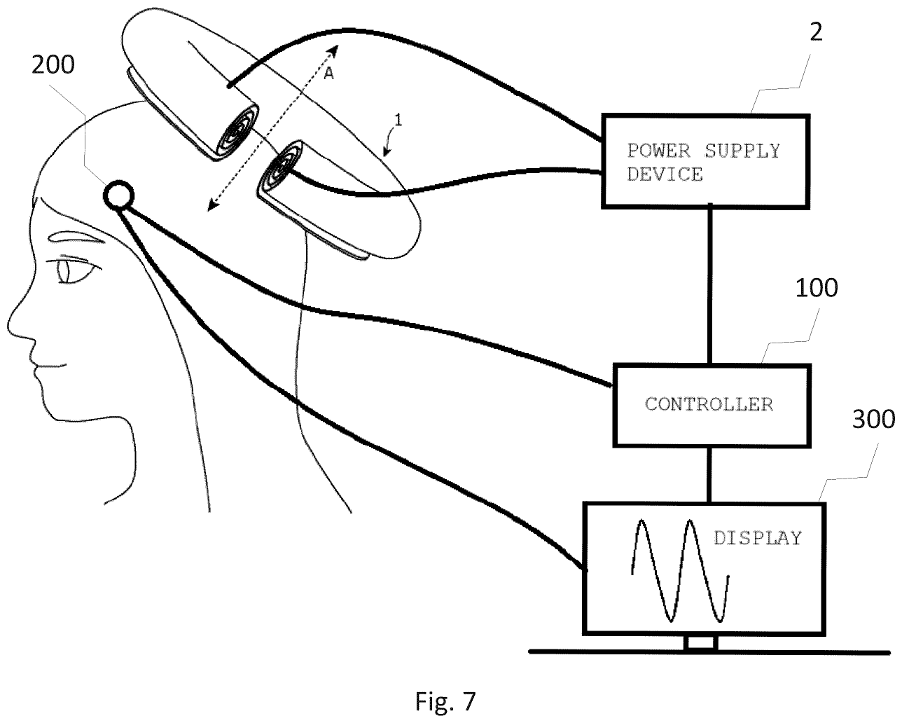

FIG. 7 is a diagram that shows an arrangement example of the vector potential generation device 1 shown in FIG. 6. For instance, in the case of the treatment device for brain diseases, such as epilepsy, brain tumors, and Parkinson's disease, as shown in, for instance, FIG. 7, the vector potential generation device 1 according to the third embodiment is arranged close to the head so that the vector potential A is applied so as to generate a strong electric field in a normal direction of the body surface.

In the case of the treatment device for the brain diseases, the treatment device may have a controller 100 that controls the power supply device 2. Further, the controller 100 may acquire and monitor an electroencephalogram (brain wave or EEG) and an electrocardiogram (EKG) of a targeted human body (from a medical equipment 200 connected to the human body) to display the acquired information on a display 300. The controller 100 may also cause the power supply device 2 to conduct the above-mentioned current to the vector potential coil device 1 at a specific timing based on the electroencephalogram and the electrocardiogram. In addition, in that case, the controller 100 may also cause the power supply device 2 to conduct the above-mentioned current to the vector potential coil device 1 in a specific pulse sequence that is effective for the treatment.

The controller 100 is configured with, for instance, a computer. The controller 100 includes at least a memory in which a program is stored and a processor such as a CPU. The processor executes the program to perform the desired operations.

Furthermore, for instance, the vector potential generation device may further have a support body in which the shape thereof matches the shape of a head of a human body, and the above-mentioned vector potential coil device 1 may be fixed to the support body. As the support body, for instance, a helmet that is worn on the head or a stand on which the vector potential coil device 1 is arranged near the head may be used. Alternatively, the above-mentioned support body may be a device that is contacted with the head, such as a pillow or a headrest of a chair. Further, in that case, the above-mentioned vector potential coil device 1 is incorporated in such the support body.

With such the support body, the vector potential coil device 1 is arranged close to a position at which a vector potential is generated in the brain of the human body. In other words, when an AC current is conducted through the vector potential coil device 1, an alternating vector potential is generated in the brain inside the head. As a result, an AC electric field or an AC current is applied to the brain. For instance, as disclosed in International Patent Publication Number WO2017/072706, brain tumors are treated by applying an AC electric field. Further, the conditions (for instance, a frequency) required for treating the brain tumors are set by the power supply device 2, and an AC electric field under such the conditions is applied non-invasively to the brain by the vector potential coil device 1. For instance, as disclosed in International Patent Publication Number WO2017/072706, in order to apply the AC electric field, electrode pads are usually attached to the skin of the head after shaving. However, according to the treatment device according to the present embodiments, shaving is not necessary, and at the same time, there is also no need to attach adhesive electrode pads to the skin of the head. As a result, burdens (physical and mental burdens) on a patient during the treatments of applying the AC electric field are reduced.

In addition, the treatment device according to the fourth embodiment can also be applicable to the diseases shown below.

(1) Diseases relating to a bone or a joint.

It may be used to treat conditions involving widespread overactive or inappropriate bone growth, such as rheumatoid arthritis, fibrodysplasia ossificans progressiva (FOP), diffuse idiopathic skeletal hyperostosis (DISH), ankylosing spondylitis, and heterotopic ossification. It may also be used for the removal of bone mass in conditions involving neoplastic bone formation or bone tumors, such as osteosarcoma, chondrosarcoma, Ewing's sarcoma, osteoblastoma, and osteoid osteoma. Similarly, it may be used to remove bone spurs that are formed in, for instance, legs, shoulders, neck, or spine as a result of chronic osteoarthritis, rheumatoid arthritis, reactive arthritis, rotator cuff injuries, plantar fasciitis, spondylosis, and/or spinal stenosis.

(2) Ligament injury.

(3) Other diseases, and the like:
(3a) Diabetes, gastritis, peptic ulcers,
ulcerative colitis, irritable bowel, and hemorrhoids;
(3b) Bronchial asthma: cold, tonsillitis, sinusitis, and chronic bronchitis;
(3c) Cardiovascular diseases: phlebitis, endarteritis, and varicose veins;
(3d) Brain and psychiatric disorders: mental disorders such as depression, aggression, anxiety, and stress, as well as Parkinson's disease, epilepsy, migraine, stroke, Alzheimer's and other degenerative brain disorders, as well as cerebral palsy, mental retardation, hyperactivity, learning disorders—by synthesizing the neurochemicals for the transmission of impulses or commands at the synaptic level and improving the electrical activity of these cells, it is possible to improve the efficiency of brain cells. In addition, it has an ability to stabilize genes and prevent an action of oxygen free radicals that are formed within cells, thus helping to slow the aging process.
(3e) Treatment of apparatus urogenitalis conditions such as menstrual irregularities, sterility, endometritis, and endometriosis in women, and orchitis, prostatitis, and oligospermia in men.
(3f) Preoperative and prophylactic treatment: VP treatments of an upper abdomen can increase blood perfusion to body extremities and reduce an inflammatory response to an injury. A preoperative preparation of a surgical site has also been shown to speed up healing.
(3g) Postoperative recovery: the symptoms of postoperative recovery nausea, motion sickness, or other forms of nausea such as vomiting can be reduced or alleviated.

(4) Use as a supplementary means.

It is a supplementary means to other treatments including at least one of cell transplantations, cultured skeleton (engineered skeleton), and growth factors. It is used to treat cartilage defects and prevent tumor metastasis.

(5) Dermatological diseases.

It can be applicable for treating skin diseases or cosmetic treatments in a living body. In particular, it can be expected to be used in the treatments of acne or hyperhidrosis, burns, and suture parts on surgically treated skin.

(6) ophthalmologic diseases.

For instance, ptosis, entropion, lagophthalmos, stye, chalazion, blepharospasm, eyelid malignant tumor, conjunctivitis, dermoid, subconjunctival hemorrhage, pinguecula, malignant lymphoma, scleritis, scleromalacia, keratitis, keratoconus, pterygium, granular corneal dystrophy, rodent corneal ulcer, uveitis, cataract, ectopia lentis, luxatio lentis, exfoliation syndrome, vitreous hemorrhage, asteroid hyalosis, persistent hyperplastic primary vitreous, glaucoma, ocular hypertension, retinal detachment, diabetic retinopathy, age-related macular degeneration, polypoidal choroidal vasculopathy, retinal artery occlusion, retinal vein occlusion, retinitis pigmentosa, retinoschisis, macular hole, epiretinal membrane, coats disease, familial exudative vitreoretinopathy, acute retinal necrosis, cytomegalovirus retinitis, retinopathy of prematurity, retinoblastoma, optic neuritis, optic nerve hypoplasia, Leber's hereditary optic neuropathy, optic canal fracture, multiple sclerosis, Devic's disease, pituitary tumor, cerebral infarction, dry eye, dacryocystitis, nasolacrimal duct obstruction, canaliculitis, myopia, hyperopia, astigmatism, presbyopia, strabismus, inferior oblique hypermobility, Duane syndrome, oculomotor paralysis, abducens nerve paralysis, Horner syndrome, Adie syndrome, traumatic mydriasis, color vision deficiency, myasthenia gravis, systemic lupus erythematosus, and thyroid eye disease.

(7) Use for non-human living bodies: it can be used for similar purposes in mammals, for instance, dogs or horses, and other non-human living bodies.

Note that various changes and modifications to the embodiments described above will be apparent to one having ordinally skill in the art. Such the changes and modifications may be made without departing from the spirit and scope of the subject matter and without diminishing the intended advantages. That is, it is intended that such the changes and modifications are included within the scope of the claims.

For instance, in any of the above-mentioned first to third embodiments, the core conductor member 12 may not be provided. Further, the power supply device 2 may be electrically connected to the above-mentioned ends 11t1 and 11t2 so as to conduct the current I(t).

In addition, in any of the above-mentioned first to third embodiments, the number of rolling times, in which the layered conductor member 11 is rolled, is not particularly limited. In addition, each of the interlayer distances of the spiral roll shape is not particularly limited, and the interlayer distances along the radial direction may be substantially constant or may vary one another.

Further, in any of the above-mentioned first to third embodiments, the electrical connection points of the power supply device 2 on the layered conductor member 11 and/or the core conductor member 12 are not limited to the above-mentioned positions, and may be positions other than the ends 11t1 and 11t2. Further, the power supply device 2 may have a plurality of electrical connection points on the layered conductor member 11 and/or the core conductor member 12 (for instance, at both ends), and in that case, the power supply device 2 is connected to each connection point by, for instance, a plurality of wirings. In addition, in the above-mentioned first to third embodiments, a material/member having a lower wire resistivity than the layered conductor member 11 may be used as the core conductor member 12, and the core conductor member 12 and the first end surface part 11a may be electrically connected at a plurality of points.

Furthermore, in any of the above-mentioned first to third embodiments, in order to ensure an insulating state between the layers of the spiral roll shape of the layered conductor member 11, an insulating coating may be applied to the surface of the layered conductor member 11, an insulating tape may be arranged on the surface of the layered conductor member 11, or a flexible board may be used as the layered conductor member 11.

Furthermore, a plurality of the vector potential coil devices 1 with linear central axes in either of the above-mentioned first and second embodiments are arranged so that their central axes are continuous, and by that, the vector potential coil devices 1 may be configured to have the coil axis of any shape (for instance, polygonal arc, polygonal ring, or spiral).

In addition, in the above-mentioned third embodiment, one end of the output terminals of the power supply device 2 may be electrically connected not to the end 12t of the core conductor member 12 but to the end 11t1 or to the end of the core conductor member 12 that is an opposite side of the end 12t so as to conduct the current I(t).

Furthermore, in the third embodiment, the layered conductor member 11 is formed in the ring shape having both side ends. However, instead of the above configuration, the layered conductor member 11 may be formed in a complete ring shape having no end (an endless ring shape). In that case, a hole may be provided in the layered conductor member 11, and the power supply device 2 may be electrically connected to the first end surface part 11a on the inner circumference side (when the core conductor member 12 is not present) (or connected to the core conductor member 12 when the core conductor member 12 is present) through the hole so as to conduct the current I(t).

Furthermore, in any of the above-mentioned first to third embodiments, the vector potential generation device may have a cooling mechanism that performs a forced cooling. In that case, for instance, the core conductor member 12 is a pipe-shaped member with a hollow portion therein (for instance, a copper pipe member having excellent thermal conductivity characteristics). Further, the cooling mechanism circulates a refrigerant (for instance, pure water, carbon dioxide, or alternative freon (fluorocarbon)) through the hollow portion so as to cool the vector potential coil device 1. In addition, a refrigerant may be circulated in the spaces between the layers of the layered conductor member 11. In that case, a pipe-shaped member (for instance, a copper pipe member having excellent thermal conductivity characteristics) for conducting the refrigerant may be provided between the layers of the layered conductor member 11.

### INDUSTRIAL APPLICABILITY

The present invention can be applicable to, for instance, a vector potential generation device.

What is claimed is:

1. A vector potential coil device comprising:
a layered conductor member in a spiral roll shape;
a first end surface part of the layered conductor member on an inner circumference side of a roll; and
a second end surface part of the layered conductor member on an outer circumference side of the roll,
wherein the layered conductor member generates a vector potential by a current flowing via the first end surface part and the second end surface part.

2. The vector potential coil device according to claim 1,
wherein the layered conductor member is a plate-shaped member.

3. The vector potential coil device according to claim 1,
wherein the layered conductor member is a mesh member.

4. The vector potential coil device according to claim 1,
wherein the layered conductor member has a spiral roll shape centered on a linear central axis.

5. The vector potential coil device according to claim 1,
wherein the layered conductor member has a spiral roll shape centered on a curved central axis.

6. The vector potential coil device according to claim 1, further comprising:
a core conductor member connected to the first end surface part along the first end surface part,
wherein a current is conducted through the core conductor member, the first end surface part, and the second end surface part.

7. The vector potential coil device according to claim 6,
wherein the core conductor member has a soft magnetic property.

8. A vector potential generation device comprising:
a vector potential coil device; and
a power supply device that conducts a current through the vector potential coil device,
wherein the vector potential coil device includes:
a layered conductor member in a spiral roll shape;
a first end surface part of the layered conductor member on an inner circumference side of a roll; and
a second end surface part of the layered conductor member on an outer circumference side of the roll,
wherein the power supply device conducts the current to the layered conductor member via the first end surface part and the second end surface part to generate a vector potential in the layered conductor member.

9. A treatment device comprising:
the vector potential generation device according to claim 8; and
a controller that controls the power supply device,
wherein the vector potential coil device applies a vector potential to a living body.
